(19) United States
(12) Patent Application Publication
(10) Pub. No.: US 2005/0048462 A1
(43) Pub. Date: Mar. 3, 2005

(54) Markers and screens

(76) Inventors: Manuel Ackermann, Oensingen (CH); Andrew Ian Matus, Oberwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/484,728

(22) PCT Filed: Aug. 12, 2002
(86) PCT No.: PCT/GB02/03719

(30) Foreign Application Priority Data
Aug. 10, 2001 (GB) 0119555.1

(51) Int. Cl. C12Q 1/00; G01N 33/53; G01N 33/567

(52) U.S. Cl. 435/4; 435/7.2

(57) ABSTRACT

Provided are in vivo and in vitro methods for identifying or detecting a synapse which has been activated, or assessing the level of activation of a synapse, which method comprises: (i) determining the presence and\or amount, in a morphologically specialised postsynaptic site in the synapse (e.g. a dendritic spine), of a detectable cellular component associated with the activation (which is a ‘tag’ or ‘marker’ for the activation e.g. an actin-cytoskeleton interacting protein such as profilin II or gelsolin), and (ii) correlating the result of the determination with synaptic activation. Such assays can be useful in identifying processes involved in LTP, and also more generally in identifying modulators of synaptic activation or transmission, and hence cognitive function.

### TECHNICAL FIELD

The present invention relates generally to methods and materials for use in markers of synaptic activity and use of these for identifying modulators of such activity.

### BACKGROUND ART

It is known that experimental manipulations of experience produce changes in the shape and number of dendritic spines that form the postsynaptic contact sites for excitatory synapses in the brain (1).

A wide variety of ideas about the function of dendritic spines have been proposed. These include that they exist solely to increase the surface area of the neuron available for receiving synaptic contacts, that their role is to protect neurons from excessive excitation, that they act as electrophysiological compartments independent of the dendrite or as biochemical compartments. Evidence that the numbers or shapes of dendritic spines can change according to the sensory input of animals during development or with an animal's behavioural status has led to the proposal that they may be involved in learning and memory (2). Recent live cell imaging studies support this hypothesis by showing both spontaneous and activity-dependent changes in spine shape and number (3-6). This morphological plasticity is associated with high concentrations of actin in spines (7-9) and the appearance of actin-rich, motile protrusions on the surfaces of spine heads (3, 4). Drugs that inhibit actin dynamics block dendritic spine motility (3, 4) and also interfere with the maintenance phase of long-term potentiation (LTP) (10, 11) suggesting that actin may serve as a link between activity-induced modulation of synaptic transmission and long-term changes in synaptic morphology associated with memory consolidation. Nonetheless, actin itself is already present in spines even prior to activation—although some spines acquire more actin when “activated”, at other spines it stays the same and in others it declines.

It is known in the art that there is a mismatch in timing between the electrophysiological effects of LTP, which appear immediately, and the associated morphological changes, which require some 30 min to develop. Indeed this temporal gap is one element in a general puzzle concerning the relationship between the electrophysiological signs of synaptic plasticity and the encoding of long term memory (38, 40) namely, how is synaptic specificity maintained during the consolidation process that turns an initially labile memory into an enduring long-term memory trace? It seems that some record of prior activity is needed in order to determine whether a particular pattern of synaptic activation will give a short-lasting or long-lasting LTP i.e. the setting of a “tag” to mark an activated synapse as the future recipient of the consolidation process (38, 41), possibly by sequestering plasticity related proteins. However, although such a distinctive tag has been mooted (41) it has not been identified to date.

Naisbitt et al. (1999) Neuron 23: 569-582 describes ‘Shank’, a protein that binds to NMDA receptor complexes. This publication asserts that when synapses are activated by glutamate, the co-localization of shank and cortactin increases from about 5 to 25%. However both shank and cortactin are present in the synapse in both stimulated and unstimulated states and therefore their presence within the synapse is not per se a tag for activation.

Ehlers (2000) Neuron 28: 511-525 refers to glutamate receptors (AMPA receptors; AMPAR) being present at high concentrations at the postsynaptic membrane. Whereas synaptic stimulation with glutamate results in rapid loss of AMPARs, induction of LTP causes translocation of AMPAR subunit GluR1 into dendritic spines and synapses. However the fact that the AMPA receptors turn over at the synapse suggests that they are not durable markers of activation and may in any case be present in even non-activated mature synapses.

It is therefore of considerable interest in the art to be able to readily identify synapses which have been activated with a “tag” which is a durable marker, the detectable presence of which in the synapse is closely correlated with the activation thereof. Using electrophysiological approaches only very small numbers of neurons (typically 60) can be studied simultaneously and individual synapses lie below the level of resolution. However the identification of a marker tag would permit the identification of populations of synapses involved in conducting a particular patter of stimuli through a neural network either in vitro or in vivo. It would also, inter alia, facilitate the identification of compounds which modulate synaptic activation, and hence downstream neurochemical and neurophysiological events.

After the presently claimed priority date, Murase et al (2002, Neuron 35: 91-105) discussed activity-induced changes in β-catenin, which moves from dendritic shafts into spines upon depolarization, apparently influencing synaptic size and strength. This molecule mediates interactions between cadherins and the actin cytoskeleton.

### DISCLOSURE OF THE INVENTION

The present inventors have investigated the activation of spine-bearing hippocampal neurons. Using live imaging of cells expressing fluorescently labelled proteins, the inventors showed that activation of NMDA receptors produced long-duration Ca²⁺-dependent targeting of profilin II to spine heads. Simultaneous visualization of YFP-tagged profilin II and CFP-tagged actin showed that, following its NMDA receptor-induced transport into dendritic spines, profilin II does not co-distribute with actin but is targeted to punctate sites at the surface of dendritic spines. Antibody staining showed further that these sites are aligned with clusters of NMDA receptors which previous studies have shown are closely associated with the junctional region of the postsynaptic membrane in vivo (26, 30) and in vitro (8, 31). Together these data indicate that NMDA receptor activation targets profilin II to postsynaptic junctional sites at spine synapses. The primarily postsynaptic distribution of profilins observed here differs from previous immunohistochemical studies which suggested an association of profilin with presynaptic sites (28)

Following its recruitment, profilin remains concentrated in spines for many hours thus marking activated synapses. These results suggest a molecular mechanism for long-term modulation of the actin cytoskeleton in dendritic spines. Since profilin II is not detectably present at the synapse in the unstimulated state but is present in the synapse in the stimulated state it acts as a single protein marker or “tag” for synapses activated, for instance via NMDA glutamatergic receptors in particular. Further results on developing neurons before dendritic spines have formed (in culture for ca. 12 days) have shown that profilin II-GFP also targets to postsynaptic sites on the dendrite shafts when NMDA receptors are stimulated. This demonstrates that profilin II-GFP marks the postsynaptic sites of activated glutamatergic synapses (and not only those with dendritic spines).

Thus, in general terms, the present invention concerns the use of a detectable cellular component, the concentration of which increases in morphologically specialised neuronal postsynaptic sites such as dendritic spines, as a specific ‘marker’ for synaptic activation.

In one aspect there is provided a method for identifying or detecting a synapse which has been activated, or assessing the level of activation of a synapse, which method comprises:
(i) determining the presence and\or amount in a morphologically specialised postsynaptic site in the synapse of a detectable cellular component associated with the activation, and
(ii) correlating the result of the determination with synaptic activation.

By “synapse” (and hence synaptic) is meant the structure where synaptic transmission occurs between two neurons, consisting of morphologically specialized presynaptic and postsynaptic components which are joined at a synaptic junction.

By “activation” (and hence activated) is meant that the synapse has been implicated in an adaptive change in synaptic transmission or connectivity in response to a particular pattern of stimulation such as repetitive patterns of stimulation known to induce LTP (36-38) or long term depression (LTD) (see Bear, M. F. (1996). A synaptic basis for memory storage in the cerebral cortex, Proc Natl Acad Sci USA 93, 13453-9.). Adaptive changes include, without limitation, morphological changes of the synapse and molecular rearrangement of structural proteins related to the actin cytoskeleton (8, 9). The changes, in vivo, may be related to cognition or processing of sensory information. Activation may be achieved, under appropriate conditions, by stimulation of post-synaptic receptors with the result that Ca²⁺ flows into a spine forming part of the synapse.

The determination step in the method may be preceded by exposing the synapse to a putative or known activating stimulus, including pharmacological agents or electrical stimuli.

The method thus encompasses the identification of synapses which have been activated by means of a detectable tag or marker which is “associated” with the activation in that its presence and\or amount in the synapse is increased in activated synapses. In general the marker, under the conditions used in the method, will not be present (or detectably present) prior to activation, but the amount of the marker in the synapse will actually durably increase to detectable levels when the synapse is activated e.g. such that detectable levels are present for at least, for example, 1, 5, 10, most preferably 15 hours or more. The “determination” of the presence and\or amount of the detectable cellular component may be either qualitative or quantitative, and the “correlation” may be based on comparison with the marker in unactivated synapses (either directly, or based on historical or contemporaneous comparison) For instance the amount of detectable cellular component may be scored in each and the scores compared.

Some embodiments by which this and other aspects of the present invention may be practised will now be discussed in more detail.

Choice and Use of Marker

As described above the present inventors have demonstrated in particular that profilin II is a suitable marker or tag. Profilin is a small protein that binds actin monomers (12) and enhances addition of G-actin to fast growing barbed ends of actin filaments at the cell surface (13, 14). Binding of profilin to a variety of proline-rich proteins and to PIP₂ is thought to promote recruitment of monomeric actin to sites of high actin dynamics at the cell surface and regulate its polymerization (15-17). Profilin II is almost exclusively expressed in the brain where it is several-fold more abundant than more widely expressed profilin I (19, 20). However a role for profilin II as a marker for synaptic activation has not previously been suggested. The combination of targeting and persistence shown by profilin II make it a potentially valuable histochemical tag for activated synapses, and the first such marker to be described.

Since dynamic actin is required for the long duration effects of LTP (11), in the light of the present disclosure, it appears that the role of profilin II as a tag for activation is likely to be related to a role in modifying the specialized actin-based cytoskeleton in dendritic spines. This cytoskeleton involves actin-regulating proteins as well as actin itself (8, 9, 29) and such proteins e.g. gelsolin, cofilin, ADF (actin depolymerizing factor) and other actin regulating proteins (i.e. as described above, those actin-related proteins capable of modifying the actin cytoskeleton in dendritic spines) may also have utility in the present invention. Results demonstrating use of gelsolin as a marker are described hereinafter.

Indeed the identification of profilin II as a synaptic tag can facilitate the identification of other tags as described below.

In order to be used in the present invention the marker or tag must be detectable. In principle it may be inherently detectable (e.g. a protein identifiable by a consonant antibody). Immunological methods of detection include immunohistochemical staining of cells or tissue sections, and assay of cell culture, to quantitate directly the expression of gene product. Antibodies useful for immunohistochemical staining and/or assay of sample fluids may be either monoclonal or polyclonal, and may be prepared by conventional methods. Conveniently, the antibodies may be prepared against a native sequence of the marker.

In preferred embodiments the marker will include a specific detectable label, which may be a ‘signal protein’, for increased ease of scoring and/or sensitivity. Most preferably the activity of the signal protein, or the protein itself, can be estimated photometrically (especially by fluorimetry or luminometry). This may be directly e.g. using for instance green fluorescent protein, insect luciferase, and photobacterial luciferase. Alternatively it may be indirect e.g. whereby the signal gene causes a change which is detected by a colour indicator e.g. on staining. Other suitable signal proteins (which have a readily detectable activity) are known in the art e.g. β-galactosidase, which can generate a coloured product from its substrate. The signal may utilise co-factors.

The invention further provides methods of increasing the detectability of a synapse on activation, which method comprises introducing into one or more neurons forming the synapse a detectable cellular component, the presence and\or amount of which is increased in a morphologically specialised postsynaptic site in the synapse in response to activation.

Thus the invention embraces methods for identifying or detecting a synapse which has been activated, or assessing the level of activation of a synapse, which methods comprises (i) introducing into one or more neurons forming a synapse a detectable cellular component, the presence and\or amount of which is increased in a morphologically specialised postsynaptic site in the synapse in response to activation, and (ii) determining the presence and\or amount of the cellular component in the postsynaptic site in the synapse, and (iii) correlating the result of the determination with synaptic activation.

The labelled marker will generally be employed by introducing it into the neurons forming the synapse (e.g. by expression from nucleic acid encoding therefore) such that on activation it can be recruited into the synapse. There is no requirement that labelled marker of the present invention introduced to the synapse have to include the full-length “authentic” sequence of the marker protein as it occurs in nature. Variants may be used (e.g. which are derived from profilin II) which retain its activity as a tag. The term “derived” includes variants produced by modification of the authentic native sequence e.g. by introducing changes into the full-length or part-length sequence, for example substitutions, insertions, and/or deletions. This may be achieved by any appropriate technique, including restriction of the coding sequence with an endonuclease followed by the insertion of a selected base sequence (using linkers if required) and ligation. Also possible is PCR-mediated mutagenesis using mutant primers. It may, for instance, be preferable to add in or remove restriction sites in order to facilitate further cloning.

Modified sequences according to the present invention may have a sequence at least 70% identical to the sequence of the marker. Typically there would be 80% or more, 90% or more 95% or more or 98% or more identity between the modified sequence and the authentic sequence. There may be up to five, for example up to ten or up to twenty or more nucleotide or amino acid deletions, insertions and/or substitutions made to the full-length or part length sequence provided functionality is not totally lost. Similarity or identity may be as defined and determined by the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Preferably sequence comparisons are made using FASTA and FASTP (see Pearson & Lipman, 1988. Methods in Enzymology 183: 63-98). Parameters are preferably set, using the default matrix, as follows: Gapopen (penalty for the first residue in a gap): −16 for DNA; Gapext (penalty for additional residues in a gap): −4 for DNA KTUP word length: 6 for DNA. Alternatively, homology in the context of nucleic acids can be judged by probing under appropriate stringency conditions. One common formula for calculating the stringency conditions required to achieve hybridization between nucleic acid molecules of a specified sequence homology is (Sambrook et al., 1989): Tₘ=81.5° C. +16.6 Log [Na+]+0.41 (% G+C)−0.63 (% formamide)−600/#bp in duplex.

Methods for introducing the labelled marker onto synapses are discussed in more detail below.

Synapses

Generally speaking, in the methods of the present invention, one or more of a population of synapses may be assessed (and optionally compared).

As described above, a synapse may be any structure where synaptic transmission occurs between two neurons, the synapse consisting of morphologically specialized presynaptic and postsynaptic components which are joined at a synaptic junction.

In the adult brain more than 90% of excitatory synapses—which use glutamate as the neurotransmitter substance—are made onto dendritic spines. Typically this is a small protrusion from the dendrite, ca. 1 micron long, consisting of an expanded head attached to the dendrite by a thin neck. The presynaptic bouton contacts the spine on the head through a synaptic junction where transmission occurs.

The invention may be carried out in various in vivo or in vitro formats as described below.

Related Methods

Methods of the present invention may be used to determine whether a synapse is activated.

As described above, activation is associated with actin reorganisation i.e. generally speaking profilin II targeting alters actin function in the spine (i.e. modulates the spine cytoskeleton and hence synaptic structure). In principle the method may be used to determine such altered actin function. The altered function may correspond to stabilization (i.e. decreased dynamic).

Several features of the activity-dependent targeting of profilin II (and gelsolin) into spines suggest a relationship to long-term potentiation (LTP), a cellular model for learning and memory in the hippocampus (36-38). Like the targeting of profilin II to spines, the induction of LTP depends on the activation of postsynaptic NMDA receptors and the influx of Ca²⁺ through receptor-associated channels. Over longer periods NMDA receptor-dependent LTP has been linked to the appearance of new spines (5, 39).

As shown in the Examples below, profilin II (and gelsolin) targeting to spines coincides with both this long-term phase of stimulation induced activation and the correlated events of spine growth (5, 37, 39). The persistence of this actin assembly promoting protein in spines many hours after the initial stimulus has been withdrawn are compatible with a role in the long-term morphological consequences of synapse activation thought to be important for memory formation.

The invention further provides methods for determining whether LTP is activated in a synapse comprising performing a method as described herein. Likewise there are provided methods for determining whether a synapse or group of synapses are involved in LTP and\or leaning or memory.

Cell Based Formats

In preferred forms the present invention is performed by detecting the marker in a dendritic spine e.g. of a spine-bearing hippocampal neuron. For example, cultured neurons may be used. When such neurons are cultured for 3 to 4 weeks most excitatory synapses are made onto dendritic spines like those in adult brain.

In a further embodiment, developing neurons may be used before dendritic spines have formed (in culture for ca. 12 days). In such cases the detectable marker (e.g. profilin II-GFP) is still targeted to postsynaptic sites on the dendrite shafts when NMDA receptors are stimulated i.e. profilin II-GFP for example marks the postsynaptic sites of activated glutamatergic synapses (and not only those with dendritic spines).

In order to produce suitable test systems including detectable markers, it will generally be preferred to use nucleic acid encoding such markers which are either stably or transiently expressed.

Nucleic acids of, or for use in, the present invention (e.g. encoding labelled profilin II) may be provided isolated and/or purified from their natural environment, in substantially pure or homogeneous form, or free or substantially free of other nucleic acids of the species of origin. Where used herein, the term “isolated” encompasses all of these possibilities.

Nucleic acid according to the present invention may be in the form of, or derived from, cDNA, RNA, genomic DNA and modified nucleic acids or nucleic acid analogs.

Thus the invention also relates, in a further aspect, to use of a nucleic acid molecule which comprises a nucleotide sequence encoding the marker described above linked to a detectable label, in the various methods of the invention.

Nucleic acid sequences which encode a marker polypeptide or peptide linked to a label in accordance with the present invention can be readily prepared by the skilled person using the information and references contained herein and techniques known in the art (for example, see Sambrook, Fritsch and Maniatis, “Molecular Cloning, A Laboratory Manual”, Cold Spring Harbor Laboratory Press, 1989, and Ausubel et al., Short Protocols in Molecular Biology, John Wiley and Sons, 1992). These techniques include (i) the use of the polymerase chain reaction (PCR) to amplify samples of the relevant nucleic acid, e.g. from genomic sources, (ii) chemical synthesis, or (iii) preparation of cDNA sequences.

Thus in cell-based assay embodiments of the present invention, the labelled marker of interest can be introduced by causing or allowing the expression in a cell of an expression construct or vector.

The construct may include any other regulatory sequences or structural elements as would commonly be included in such a system, and as is described below. As well as the signal sequence, the vector components will usually include, but are not limited to, one or more of an origin of replication, one or more marker genes, an enhancer element, a promoter, and a transcription termination sequence. Construction of suitable vectors containing one or more of these components employs standard ligation techniques which are known to the skilled artisan.

Nucleic acid sequences which enable a vector to replicate in one or more selected host cells are well known for a variety of bacteria, yeast, and viruses. For Example, various viral origins (SV40, polyoma, adenovirus, VSV or BPV) are useful for cloning vectors in mammalian cells.

Particularly preferred is an expression vector comprising a nucleic acid as described herein. The vector may, for example, be in the form of a plasmid, cosmid, viral particle, phage, or any other suitable vector or construct which can be taken up by a cell and used to express the detectable marker.

Expression vectors usually contain a promoter which is operably linked to the protein-encoding nucleic acid sequence of interest, so as to direct mRNA synthesis. Promoters recognized by a variety of potential host cells are well known. “Operably linked” means joined as part of the same nucleic acid molecule, suitably positioned and oriented for transcription to be initiated from the promoter. DNA operably linked to a promoter is “under transcriptional control” of the promoter. Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g. the actin promoter or an immunoglobulin promoter, and from heat-shock promoters, provided such promoters are compatible with the host cell systems.

Expression vectors of the invention may also contain one or more selection genes. Typical selection genes encode proteins that (a) confer resistance to antibiotics or other toxins e.g. ampicillin, neomycin, methotrexate, or tetracycline, (b) complement auxotrophic deficiencies, or (c) supply critical nutrients not available from complex media, e.g., the gene encoding D-alanine racemase for Bacilli.

The method referred to above may therefore further include introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For example, the calcium phosphate precipitation method of Graham and van der Eb, Virology 52:456-457 (1978) can be employed. General aspects of mammalian cell host system transformations have been described in U.S. Pat. No. 4,399,216. For various techniques for transforming mammalian cells, see Keown et al., Methods in Enzymology, 185:527 537 (1990) and Mansour et al., Nature 336:348-352 (1988).

Host cells transfected or transformed with expression or cloning vectors described herein may be cultured in conventional nutrient media. The culture conditions, such as media, temperature, pH and the like, can be selected by the skilled artisan without undue experimentation. In general, principles, protocols, and practical techniques for maximizing the productivity of cell cultures can be found in “Mammalian Cell Biotechnology: a Practical Approach”, M. Butler, ed. JRL Press, (1991) and Sambrook et al, supra.

In Vivo and Animal Formats

Host cells according to the present invention (i.e. including the detectable marker) may be comprised in a transgenic animal, and the present invention further provides for a transgenic animal, comprising cells which express a labelled marker according to a preceding aspect, such as a fluorescent form of profilin II, and also uses thereof.

The transgenic organisms of the invention all include within a plurality of their cells a cloned recombinant or synthetic DNA sequence which encodes the labelled marker.

Since it is possible to produce transgenic organisms of the invention utilizing a variety of labelled markers, a general description will be given of the production of transgenic organisms by referring generally to exogenous genetic material. This general description can be adapted by those skilled in the art in order to incorporate the above-described specific DNA sequences into organisms and obtain expression of those sequences utilizing the methods and materials described below. For more details regarding the production of transgenic organisms, and specifically transgenic mice, refer to U.S. Pat. No. 4,873,191, issued Oct. 10, 1989 (incorporated herein by reference to disclose methods producing transgenic mice), and to the numerous scientific publications referred to and cited therein.

The exogenous genetic material may be placed in either the male or female pronucleus of the zygote. More preferably, it is placed in the male pronucleus as soon as possible after the sperm enters the egg. In other words, right after the formation of the male pronucleus when the pronuclei are clearly defined and are well separated, each being located near the zygote membrane. The male pronucleus of a fertilized mouse egg is the preferred site for addition of the exogenous genetic material of the present invention.

It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material could then be added to the ovum or the decondensed sperm could be added to the ovum with the exogenous genetic material being added as soon as possible thereafter.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the DNA sequences which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of a gene, in order to insure that one copy is functional. As regards the present invention, there is generally an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Thus the present invention provides methods in which cloned recombinant DNA sequences encoding appropriate membrane targeting sequences may be injected into fertilized mammalian eggs (preferably mouse eggs). The injected eggs are implanted in pseudo pregnant females and are grown to term to provide transgenic mice whose cells express proteins related to the pathology of the relevant disease. The injected sequences are constructed having promoter sequences connected so as to express the desired protein in specific tissues of the transgenic mammal (most notably in nerve tissue). Examples may include the prion specific promoter of Lewis et al (2000) Nature Genetics 25, 402-405, or the neurospecific enolase promoter.

Non-human animals of the invention may be homozygous or heterozygous for the fusion polypeptide. Mammalian animals include non-human primates, rodents, rabbits, sheep, cattle, goats, pigs. Rodents include mice, rats, and guinea pigs. Specifically provided are:

Methods of preparing a transgenic animal model in which activation of synapses (and hence the presence of synapses involved in LTP and memory formation) can be readily detected, optionally following defined stimuli or treatment,

Methods of producing an F₁ generation by crossing a founder animal of either sex (F₀ generation) with an animal which is non-transgenic in respect of the proteins discussed herein, and is preferably wild-type). The offspring (F₁ generation) may then be screened and those which carry a transgene as above, are selected. Methods of producing an F₂ generation by crossing 2 F₁ animals of appropriate sex. The offspring (F₂ generation) may then be screened and those which carry a transgene in the appropriate dosage (i.e. hetero or homozygous), are selected.

A method of enhancing the detectability of activation of a synapse in a transgenic animal model, and the transgenic organism with the modulated phenotype, as described above.

For example, the invention provides profilin II-GFP mice the brains of which may be used to study the “tagging” of synapses. Cell cultures from brain tissue of these mice show that the same glutamate receptor-dependent targeting characterized in the transfected cells (discussed in more detail in the Examples hereinafter) also occurs in brain neurons from the mice. Such mice are normally-behaving animals whereby the expression of labelled-tag is regulated at levels that do not interfere with function. The fact that the activity-dependent targeting happens in the mouse tissue is thus a good confirmation of the physiological relevance of the effect.

Transgenic non-human mammals of the invention may, inter alia, be used for experimental purposes in studying neuronal activation and the development of therapies designed to alleviate the symptoms of disease in which improper activation is implicated. By “experimental” it is meant permissible for use in animal experimentation or testing purposes under prevailing legislation applicable to the research facility where such experimentation occurs.

Modulators of Synaptic Activation

An important aspect of the present invention is the use of the detectable markers disclosed herein to screen for modulators affecting synaptic transmission, for example modulators (e.g. agonists or antagonists) affecting cognitive function (memory and learning, including drug addiction), epilepsy, neurodegeneration, ischemia, migraine, schizophrenia and depression. This can be achieved in the light of the disclosure herein by looking for compounds that would enhance (for agonists) or supress (for antagonists) marker localizing to spines (or cause redistribution of other proteins that regulate the actin cytoskeleton to or from spines). Thus, an indicator for activated synapses is useful for screening for compounds that improve long term memory, important for diseases such as Alzheimer's. As shown in the Examples below, profilin II stays in the synapse for at least 16 hours and hence marks activated synapses over a sufficient period to screen for the effect of modulators.

Accordingly, the present invention provides a method for screening compounds for the potential modulation of cognitive function comprising assaying for a modification of labelled marker targeting to morphologically specialised postsynaptic site in synapses.

The term “cognitive function” for the purposes of the present invention in particular includes cortical functions of the brain. Consciousness and memory therefore are examples of “cognitive function”. Various conditions in the human, including diseases and congenital diseases, may affect cognitive functions. For example, dementias such as Alzheimer's disease or mental retardation are characterized by an impaired cognitive function. Numerous tests to assess cognitive function are known to the person skilled in the art of psychology and medicine. For example, cognitive function may be assessed using psychometric tests, neuropsychological tests, metacognitive self-evaluations, cognitive screening tests and reaction time tests (Ruoppila, I. and Suutama, T. (1997) Scand. J. Soc. Med. Suppl. 53, 44-65). The term “cognitive function” includes the functions assessed by any such test. The term “modulation” of cognitive function is meant to include increasing as well as reducing cognitive function. Increase as well as reduction of cognitive function may be assessed by any of the above-mentioned tests.

Non-Neuronal Screens

Although preferred methods of screening of the present invention are based on neuronal cells or cells of neuronal origin, non-neuronal cell types displaying appropriate characteristics may be suitable cells for the purposes of the present invention.

For example, in non-neuronal cells profilin is recruited to sites on cell surfaces (13, 18, 34). Thus, in addition to the cultured or extracted neuron cells discussed above, screening (or pre-screening) for modulators of activation may be carried out e.g. with fibroblasts, optionally with transient expression of profilin II. This may be more convenient than use of neurons, and positive ‘hits’ can then be taken forward for use in neuron or whole-animal studies.

In one embodiment the present invention provides a method for screening compounds for the potential modulation of cognitive function comprising the steps of: (a) measuring profilin II translocation to actively ruffling membrane, (b) exposing such cells to a candidate compound, (c) measuring profilin II translocation to actively ruffling membrane in said cells in the presence of the candidate compound, (d) comparing profilin II translocation to actively ruffling membrane in said cells in the absence and the presence of the compound, (e) choosing a compound that modifies profilin II translocation to actively ruffling membrane.

Neuronal Screens

However, because the profilin II “tag” mechanism operates at synapses and is regulated by synaptic transmission, the method is preferably used with neuronal cells. Similarly the redistribution to the cortical cytoskeleton of a cell of the other actin-regulating proteins discussed above may be assayed in neurons.

Thus the various methods described above may comprise the further steps of contacting the synapse, or neurons forming it, with one or more agents which it is desired to assess for ability to modulate synaptic activation, and comparing the activation in the presence or absence of said agents—the relative values may be correlated with its activity as a modulator.

In one embodiment the present invention provides a method for screening compounds for the potential modulation of cognitive function comprising the steps of: (a) measuring the presence and\or amount in a morphologically specialised postsynaptic site in the synapse of a detectable marker, (b) exposing the neurons forming the synapse to a candidate compound, (c) measuring the presence and\or amount in a morphologically specialised postsynaptic site in the synapse of the detectable marker in the presence of the candidate compound, (d) comparing the determinations made in (a) and (c), (e) choosing a compound that modifies the amount in a morphologically specialised postsynaptic site in the synapse of a detectable marker.

For example a dendritic spine on a rat hippocampal neuron in culture may be visualized by a fluorescent protein (e.g. Profilin II-GFP) enriched in the spine head and may be recorded before and after perfusion with a compound (e.g. after 30 minutes in a solvent such as neuronal cell medium).

Where it is desired to screen for modulators which are inhibitors, it will generally be required to include an initiation or stimulation of activation step in the method. Thus such methods herein will generally include the step of exposing the synapse (or neurons forming the synapse, or non-neuronal cells) to a putative or known stimulus or cause of activation. Methods may also include providing other components involved in activation e.g. Ca²⁺.

For cultured neurons or other appropriate tissue samples this may be by stimulation of appropriate receptors. As shown below, stimulation of glutamate NMDA receptors may be particularly effective.

Screens Using Transgenic Animals and Derived Cells and Tissues

Tag visualization techniques may be used in intact animals (or brain tissue or neurons derived from such animals) e.g. in disease models, after drug treatment or following behavioural manipulation.

Methods utilising physiological stimulation, in particular electrical stimulation, to induce the release of neurotransmitter and its subsequent interaction with its cognate receptor may be particularly preferred in cases where it is desired to avoid exposing cells to glutamate to get targeting. Alternatively, LTP or LTD can be induced in Profilin II-GFP brain slices using appropriate electrical stimulation (LTP, tetanic stimulation at 50-100 Hz; LTD, 1 Hz) and the effect of a drug on profilin II redistribution can be determined in such systems. Alternatively, as described above, fibroblasts or blood cells could be isolated from profilin II-GFP animals to see if profilin II translocates to actively ruffling membrane under defined circumstances.

In vivo systems where a Profilin II-GFP mouse undergoes behavioural tests in the presence or absence of a test compound can also be carried out. Because profilin II remains at the synapse for at least 16 hours after activation, brain tissue can be fixed and analyzed without concern that marker distribution changes during sample preparation.

Specificity of Modulation

Where the method of identifying modulators utilizes a cell-based system, it may further include the step of testing the viability of the cells expressing the labelled marker e.g. by use of a lactate dehydrogenase assay kit (Sigma). This step may provide an indication of any interference by the test agent of vital cellular functions.

Hi-Throughput Screens

Essentially, methods of the present invention may be employed analogously to high throughput screens such as those well known in the art—see e.g. WO 200016231 (Navicyte); Wo 200014540 (Tibotec); DE 19840545 (Jerini Biotools); WO 200012755 (Higher Council for Scientific Research); WO 200012705 (Pausch MH; Wess J); WO 200011216 (Bristol-Myers Squibb); U.S. Pat. No. 6,027,873 (Genencor Intl.); DE 19835071 (Carl Zeiss; F Hoffman-La Roche); WO 200003805 (CombiChem); WO 200002899 (Biocept); Wo 200002045 (Euroscreen); U.S. Pat. No. 6,007,690 (Aclara Biosciences).

Choice of Test Compound

Compounds which are tested may be any which it is desired to assess for the relevant activity

The methods can serve either as primary screens, in order to identify new inhibitors/modulators, or as secondary screens in order to study known inhibitors/modulators in further detail.

Agents may be natural or synthetic chemical compounds. Relatively small chemical compounds, preferably which are capable of crossing the blood-brain barrier, may be preferred.

The skilled person will appreciate that the amount of test substance or compound which is added in a screening assay according to this aspect of the invention will normally be determined by trial and error depending upon the type of compound used. It may be selected to be a level which could realistically be used in therapeutic context i.e. would be non-lethal to a patient.

Typically, from about 0.01 to 100 nM concentrations of putative modulator compound may be used, for example from 0.1 to 10 nM.

Therapeutics

Performance of a screening assay method according to the various aspects above may be followed by isolation and/or manufacture and/or use of a compound, substance or molecule which tests positive for ability to interfere with or modulate the neuronal activation.

The compounds thus identified may be formulated into compositions for use in the diagnosis, prognosis or therapeutic treatment. Thus, the present invention also extends, in further aspects, to pharmaceutical formulations comprising one or more inhibitory or modulatory compound as obtainable by a screening method as provided herein.

A compound which has been identified as described above, may be manufactured and/or may be used in the preparation, i.e. the manufacture or formulation, of a composition such as a medicament, pharmaceutical composition or drug. These may be administered to individuals.

Whether it is a polypeptide, antibody, peptide, nucleic acid molecule, small molecule, mimetic or other pharmaceutically-useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16^{th} edition, Osol, A. (ed), 1980.

The agent may be administered in a localised manner to the brain or other desired site, or it may be delivered systemically in a manner such that it targets the brain or other cells.

Methods of Finding New Tags

The disclosure herein may be used to find new markers or tags of neuronal activation, for example by use of methods which include the step of detecting profilin II in the synapses, and (for example) screening for cellular components which co-localise with it, or modify the actin cytoskeleton, under activating conditions.

Such further markers (which may or may not interact directly with profilin II) may also play a role in LTP. Binding partners of profilin having these properties may be detected in two-hybrid assays e.g. as disclosed by Fields and Song, 1989, Nature 340; 245-246.

Diagnostics

Profilin II and other markers for activated synapses disclosed herein may be used as diagnostics. The pattern of activated synapses in the brain could be correlated to a particular disease state (e.g. different types of schizophrenia) and based on the diagnosis particular therapies could be chosen.

The present inventors have shown that cultured hippocampal neurons when exposed to glutamate at an excitotoxic concentration (100 microM) for 30 min showed excitotoxic changes, including loss of normal morphology and swelling of dendrites, within 2 min of exposure to glutamate. However, despite these degenerative changes, profilin II-GFP was still targeted to punctate sites within dendrites indicating that the targeting mechanism is intact. This is relevant to stroke and epilepsy where hyperstimulation of receptors by released glutamate causes neuronal damage. Thus it appears that profilin II tagging occurs even after events that ultimately lead to apoptosis have been set in motion. In toxicity models, profilin II targeting could thus act as a marker for potentially damaged cells at times before cell death occurs.

Thus the invention provides methods of diagnosis and use of the materials disclosed herein in such methods, particularly in respect of neuropathologies. The invention further provides methods of enhancing the detectability of the disease phenotype of a transgenic animal model wherein the detectability of a synapse on activation, is increased by use of detectable cellular component as described above.

The invention will now be further described with reference to the following non-limiting Figures and Examples. Other embodiments of the invention will occur to those skilled in the art in the light of these.

### FIGURES

FIG. 1: Distribution of GFP tagged profilins and GFP in dendrites of transfected hippocampal pyramidal neurons. (A). In a proportion of neurons (see Table 1) profilin II-GFP is highly enriched in dendritic spines (arrowheads) compared to the dendrite shaft. (B). In other cells profilin II was not enriched in spines. (C). Similarly GFP-tagged profilin I shows little selectivity for spines. (D). A cell expressing unmodified GFP to show to the distribution of a soluble marker between dendritic shaft and spine. Scale bar: C, 10 μm.

FIG. 2: Glutamate receptor stimulation redistributes profilins to dendritic spines. (A). Profilin II-GFP distribution in dendrites of a transfected cell before (0′) and after (30′) after exposure to 10 μM glutamate for 30 min. (B). Higher magnification detail of profilin II targeting to spine heads induced by this treatment. (B′). Detail of the boxed area from (B). (C, C′). Dendrite segment from control cell expressing GFP where glutamate treatment does not induce protein accumulation in spines. (D, D′). GFP-tagged profilin I also redistributes to spines although less prominently than profilin II. Scale bars: A, 20 μm; B, 5 μm; B′, 1 μm.

FIG. 3: Presynaptically released glutamate triggers targeting of profilin II to dendritic spines. (A, B). Profilin II-GFP distribution before (A) and after (B) 30 min in reduced extracellular Mg²⁺ (0.5 mM) showing a strong shift to spines. (C, D).

Profilin II-GFP distribution in cells exposed to zero Mg²⁺ for 30 min after being preincubated in botulinum toxin for 16 hours. There was no shift of profilin II to spines. (E). To show that the postsynaptic receptor-dependent profilin targeting mechanism was intact after botulinum toxin treatment, the same culture was exposed to 5 μM NMDA for a further 30 min. This induced strong targeting of profilin II to spines (E). Scale bars: 5 μM (A, E)

FIG. 4: Profilin II is targeted to the distal sites on dendritic spine heads. (A, B) Original gray scale data for YFP-profilin II (A) and CFP-actin (B) from a doubly transfected cell. For clarity the signals have been inverted so that high fluorescence shows dark and low fluorescence light. Arrowheads in panel A and C indicate spines with surface-located double profilin puncta. (C). Overlay image of YFP-tagged profilin II (yellow) and CFP-tagged actin (blue). Areas of overlap between the two signals appear pale green. (D, E). The two spines indicated in panels A and C at higher magnification. Each row contains (from left) an overlay image followed by inverted gray-scale data for profilin II and actin. Scale bars: A, 10 μm; D, E, 1 μm.

FIG. 5: Profilin is co-distributed with NMDA receptor clusters on spine heads. (A) Overlay image as in FIG. 4C showing profilin II-YFP and CFP-actin in 3 spines of a doubly transfected cell. (B) Inverted gray scale data for actin. The dotted lines indicate the course of the dendrite shaft. (C) Inverted gray scale data showing profilin II distribution in the same spines. (D) The same area of dendrite after fixation and staining with anti-NR1 showing the co-incident distributions of profilin II and NMDA receptors (compare panels C and D). Scale bar: A, 1 μm.

FIG. 6: Profilin II remains in spine heads after withdrawal of the targeting stimulus. (A) A dendrite segment before stimulation. Letters next to boxes identify individual spines shown in detail in panels D, E and F. (B) The same dendrite after 30 min treatment with 5 μM glutamate showing targeting of profilin II to spine heads. (C) 45 min after glutamate was washed out, profilin II was still strongly concentrated in spines. (D-F) Detail of individual spines indicated in (A) immediately before glutamate treatment (D, E, F) and 45 min after washout (D′ E′, F′). Scale bars: 5 μm, (A) and 1 μm (D, E, F).

FIG. 7: Profilin II-GFP transfected neuron treated with 10 μM glutamate in Ca²⁺-free medium. To show that in the absence of extracellular Ca²⁺ glutamate-induced targeting of profilin II to spines does not occur. Scale bar: 5 μm.

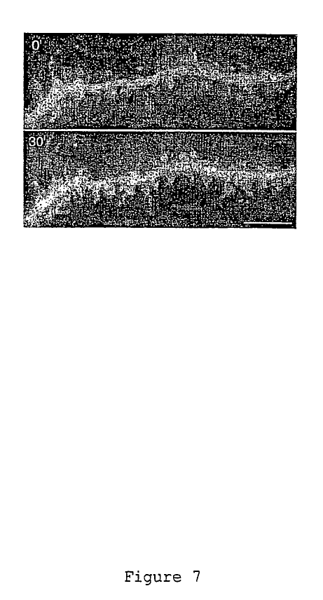

### EXAMPLES

Materials and Methods

Fusion constructs of Profilin I and II with GFP or GFP alone were expressed from a eukaryotic expression plasmid containing a β-actin promoter (42).

Active compounds were obtained from the following sources: N-methyl-D-aspartate (NMDA), amino-3-hydroxy-5-methyl-4-isoxazolepropionic acid (AMPA), (5R,10S)-(+)-5-methyl-10,11-dihydro-5H-dibenzo[a, d]cyclohepten-5,10-imine hydrogen maleate (MK-801), 6,7-dinitroquionoxaline-2,3(1 h, 4 h)-dione (DNQX), 1,2,3,4-Tetrahydro-6-nitro-2,3-dioxo-benzo[f]quinoxaline-7-sulfonamide (NBQX) D(−)-2-amino-5-phosphonopentanoic acid from RBI, (1S,3S)-1-aminocyclopentane-1,3-dicarboxylic acid ((1S,3S)-ACPD) from Alexis, (S)-3,5-dihydroxyphenylglycine ((S)-3,5-DHPG), (R, S)-□-methyl-4-carboxyphenylglycine (MCPG) and 2S-2-amino-2-(1S,2S-2-carboxycycloprop-1-yl)-3-(xanth-9-yl)propanoic acid (LY341495) from Tocris. Monoclonal antibodies to the NR1 subunit of the NMDA receptor were obtained from Pharmingen.

Cell Culture and Microscopy

Cultures of dispersed neurons (43) were transfected with cDNA constructs (3) and maintained in glia-conditioned, serum free medium for 19 to 28 days prior to imaging. Imaging was performed at 37° C. in Tyrode's solution pH 7.3, with GFP-optimized filters (Chroma Technologies, Brattleboro, Vt.) and a MicroMax cooled CCD camera (Princeton Instruments, Trenton, N.J.) controlled by Metamorph software (Universal Imaging corporation, West Chester, Pa.). Cells were stimulated by addition of glutamate or NMDA (5 to 10 μM) for up to 30 minutes. In experiments where specific receptor subtypes were blocked, cells were preincubated for 15 minutes with corresponding antagonists.

### Example 1

### Profilin I and Profilin II in Cultured Hippocampal Neurons

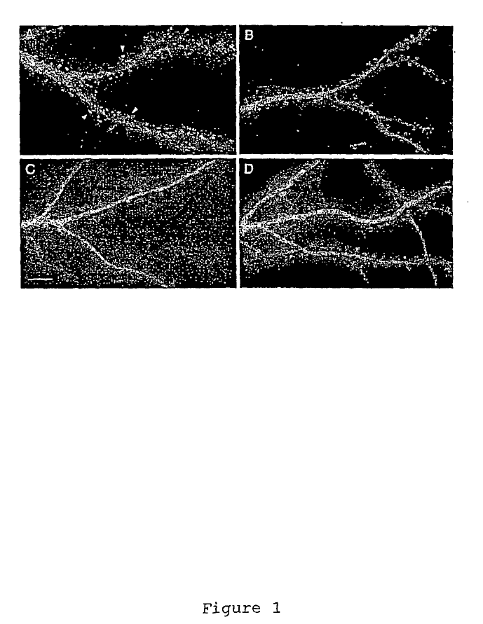

Expressing GFP-tagged profilin I and profilin II in cultured hippocampal neurons revealed differences in their distribution within dendrites (FIG. 1 and Table 1). In 4% of cells expressing profilin II (n=239) the GFP-tagged protein was highly enriched in heads of dendritic spines (FIG. 1A) whereas in others cells spine enrichment was weak or undetectable (FIG. 1B). GFP-tagged profilin I showed only weak or no enrichment in spines (FIG. 1C). For comparison the distribution of unmodified GFP in control cells is shown in FIG. 1D.

Corresponding results with gelsolin are shown in Table 5. ‘G2-6-GFP’ is a based on a gelsolin mutant which lacks the domain 1 (and hence the severing function) of whole gelsolin.

### Example 2

### NMDA Receptors Regulate Profilin II Accumulation in Spine Heads

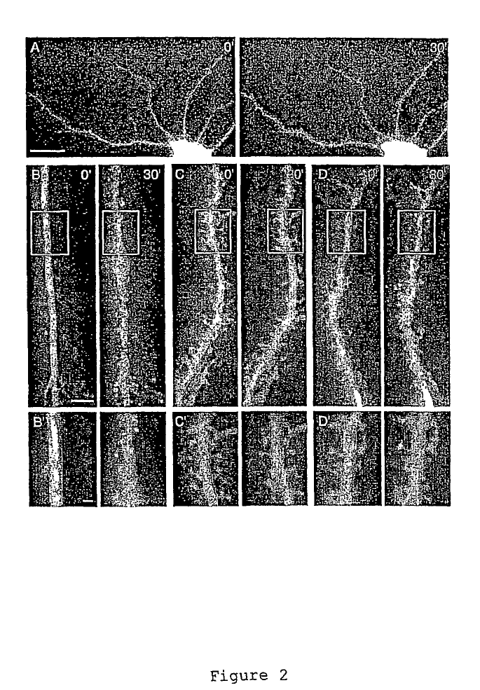

The strong enrichment of profilin II in spines of some cells but not others suggested that its cytoplasmic localization might be activity dependent. To test this hypothesis we exposed hippocampal neurons expressing GFP-tagged profilin II or profilin I or unmodified GFP to 10 μM glutamate (FIG. 2). Images of living cells captured before and after treatment showed a strong redistribution of profilin II-GFP into the heads of spines that started 5 to 8 minutes after glutamate addition and was maximal after 30 minutes (FIGS. 2A and B). Glutamate treatment also produced a weaker shift of GFP tagged profilin I into spines (FIG. 2D) which, however, never approached the high levels seen for profilin II. The same treatment did not change the distribution of unmodified GFP (FIG. 2C).

To determine which sorts of glutamate receptor might be involved in profilin targeting we treated profilin II-GFP transfected cells with a variety of subtype-selective glutamate receptor agonists and antagonists (Table 2). In control experiments, cells expressing profilin II-GFP showed strong redistribution of the protein to dendritic spines following treatment with 10 μM glutamate (Table 2a). Treatment with NMDA produced strong targeting of profilin II to spines in all cells similar to that observed for glutamate (Table 2b). However, stimulating cells with DHPG, an agonist selective for mGluR1-type metabotropic glutamate receptors, was markedly less effective, while AMPA was ineffective in 14 of 16 cells tested and showed only marginal effects in the remaining two cells. These contrasting effects of different glutamate receptors on profilin II-GFP targeting to spines were confirmed by experiments where cells were stimulated with glutamate while individual receptor subtypes were blocked by selective antagonists (Table 2c). Blocking NMDA receptors with the antagonist APV greatly reduced glutamate-induced profilin II targeting so that out of 28 cells tested none showed the strong targeting produced by treatment with glutamate alone and only 5 showed weak spine labeling. By contrast, blocking either AMPA or metabotropic glutamate receptors or both together was far less effective in inhibiting glutamate-induced accumulation of profilin II in spines.

Corresponding results with gelsolin are shown in Table 6. In these experiments only 2.5 μM glutamate was used.

### Example 3

### Increased Calcium Levels are Required for Spine Targeting of Profilin II

The involvement of NMDA receptors in the accumulation of profilin II in spines suggested that increases in cytoplasmic Ca levels may be required to trigger profilin II targeting. Consistent with this idea, profilin II levels in spines failed to increase when cells were treated with glutamate in the absence of extracellular Ca²⁺ (Table 3, see also supplementary figure). To determine whether elevation of intracellular Ca²⁺ is alone sufficient to trigger the targeting process, we treated neurons with thapsigargin (1 μM) which results in the release of Ca²⁺ from internal stores and leads to influx of extracellular Ca²⁺ (21, 22). Following thapsigargin treatment 10 out of 15 cells tested showed accumulation of profilin II in spines (Table 3). When cells were treated with thapsigargin in the absence of extracellular Ca²⁺ there was only weak redistribution of profilin II in a minority of cells (Table 3a) suggesting that the effect of thapsigargin depends mainly on Ca²⁺ entering the spine cytoplasm from outside (22) rather than release from intracellular stores.

In further experiments we exposed cells to the mGluR I agonist 1S, 3R-ACPD which stimulates a concentration-dependent release of Ca²⁺ from internal stores. At low concentrations (100 μM) this agonist provokes release only from IP₃ receptor-dependent stores leading to a modest increase in cytoplasmic Ca²⁺ concentration (23). Used at this lower concentration 1S,3R-ACPD did not produce any change in distribution of profilin II in dendrites (Table 3b). At the higher concentration of 1 mM, where 1S,3R-ACPD produces larger increases in cytoplasm Ca²⁺ levels by activating ryanodine-sensitive Ca²⁺ stores via calcium-induced calcium release (CICR) (23) it produced strong targeting of profilin II to spines. Treating cells with an intermediate concentration of 1S,3R-ACPD (500 μM) produced an intermediate result (Table 3b), underscoring the dependence of profilin II targeting on intracellular Ca²⁺ levels.

### Example 4

### Presynaptically Released Glutamate Triggers Profilin II Redistribution

Profilin II targeting to spines could also be induced by depolarizing cell through exposure to 90 mM KCl. Treating the cells continuously for 30 min produced a weak shift of profilin II into spines (n=2). A more effective procedure was to give cells spaced stimulation with 4 pulses of 90 mM KCl, each lasting for 3 min separated by 10 min recovery intervals (24). This produced reliable accumulation of profilin II in spines with strong targeting in 3 of 5 cells tested (Table 4a). In both continuous and spaced treatment the effects of depolarization with 90 mM KCl depended on activation of NMDA receptors since in the presence of APV none of the cells tested (continuous, n=5; spaced, n=7) showed any redistribution of profilin to spines.

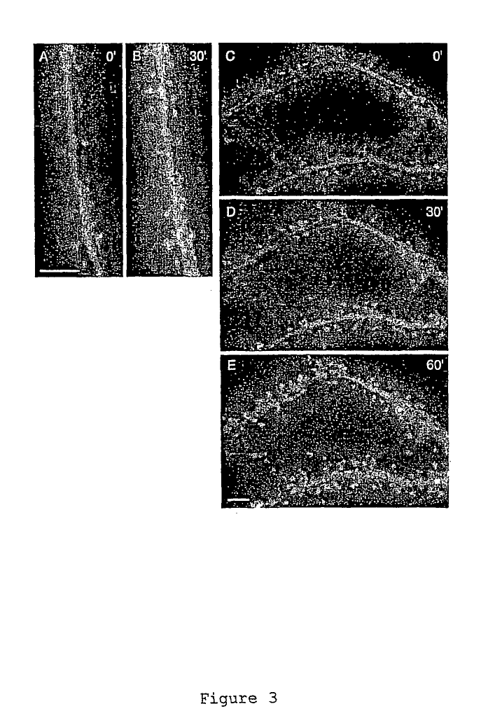

One effect of depolarization with 90 mM KCl is to activate postsynaptic NMDA receptors by relieving the Mg²⁺ block that operates on them at resting membrane potentials. To see whether this was the case we followed the distribution of profilin II in cells after removing Mg²⁺ from the medium. After 30 minutes, 11 of 12 neurons examined showed redistribution of profilin II to spine heads (FIG. 3A and Table 4). Spine targeting of profilin II following removal of extracellular Mg²⁺ did not occur if presynaptic release of neurotransmitter was blocked by preincubating neurons (n=7) with botulinum toxin (100 ng/ml) for 24 hours prior to the experiment. Subsequently treating the same cells with 5 μM NMDA produced strong targeting of profilin to spines (FIG. 3B and Table 4) showing that the postsynaptic receptor-dependent mechanism was intact after botulinum toxin treatment. Taken together these results indicate that the accumulation of profilin II in spines is triggered by synaptically released glutamate acting on postsynaptic NMDA-type glutamate receptors.

### Example 5

### Profilin is Targeted to Synaptic Junctional Sites in Dendritic Spines

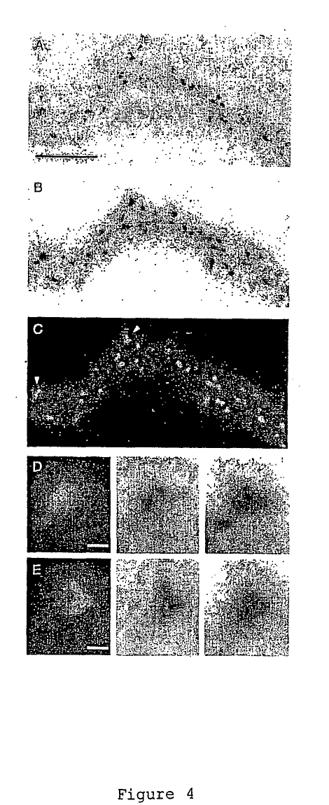

Previous studies implicate profilin in recruiting monomeric actin to sites of high motility and loading it onto the barbed end of actin filaments (13, 16). Consequently, the NMDA receptor-induced redistribution of profilin II to dendritic spines we observed suggested that it might be involved in delivering monomeric actin to sites of filament elongation within spines. To test this possibility we examined cells doubly transfected with profilin II-YFP and CFP-actin allowing us to visualize the distributions of profilin II and actin simultaneously and independently (FIG. 4). Comparing the distributions within spine heads of profilin II-YFP (FIG. 4A) and CFP-actin (FIG. 4B) suggested that the precise localization of the two molecules within the spine head was different, a conclusion that was confirmed when the two images were overlaid (FIG. 4C). This revealed that profilin II was concentrated at the tips of spine heads in one or more distinct spots (FIGS. 4A and C, arrowheads). By contrast, actin was distributed throughout the cytoplasm in all spine heads (FIGS. 4B and C).

This distribution of profilin II suggested that it might be associated with postsynaptic junctional sites which are similarly distributed at the tips of dendritic spines at one or more discrete locations (25).

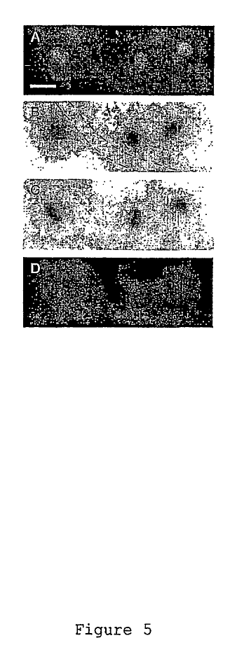

We accordingly compared the distribution of profilin II-YFP to that of antibody-stained NR1 subunits of NMDA receptors which are known to be closely associated with postsynaptic junctional sites (26). This showed a striking colocalization of profilin II puncta and NR1 clusters at the tips of spines in comparison to the actin which was distributed throughout the spine cytoplasm (FIG. 5).

### Example 6

### Glutamate Receptor-Induced Accumulation of Profilin II in Spines Heads Endures for Hours

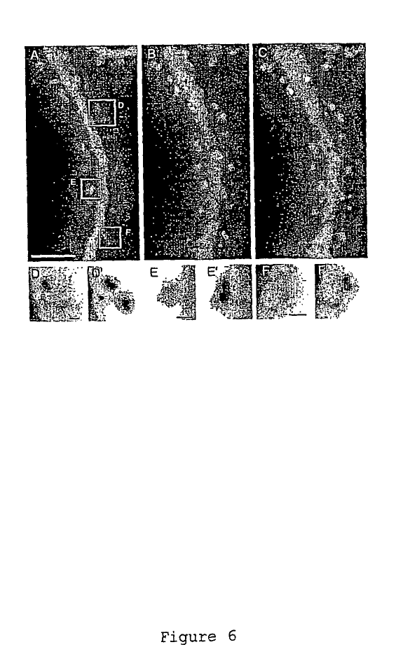

We next addressed the question of how long profilin II remains localized in spine heads following its NMDA receptor-dependent accumulation there. As before, cells expressing GFP-tagged profilin II were treated with glutamate in Tyrode's solution for 30 min to induce profilin II targeting, then washed with 4 changes of Tyrode's solution alone. The cells were observed after a further 45 min “recovery” period. At the end of this time there was no reduction in profilin II-GFP levels in the spines (FIG. 6). Subsequently we stimulated cells with glutamate for 30 min to induce profilin II accumulation in spine heads and re-examined them after 4 or 16 hours of further incubation in culture medium without glutamate. Even after these long periods of post-stimulus incubation profilin II remained concentrated in spine heads at undiminished levels (n=10 cells at 4 h, 4 cells at 16 h).

### Example 7

### Transgenic Mice

Transgenic mice were prepared using conventional methodology as described by Hogan, B., Beddington, R., Costantini, F., and Lacy, E. (1994). Manipulating the mouse embryo, 2nd edn (Plainview, N.Y., Cold Spring Harbor Laboratory Press) and summarised earlier herein.

Briefly, 10 micrograms of DNA containing a 3 kb chicken beta-actin promoter together with the coding sequence of profilin II fused to the coding suquence of green fluorescent protein was purified on an Elutip column (Schleicher and Schuell, Dassel, Germany) and dissolved at 2.5 micrograms per milliliter in injection buffer (5 mM tris buffer (pH 7.4) and 0.5 mM EDTA). Fertilized eggs for injection were obtained from mice produced by crossing male C57BL/6 with females of the F1 generation from a cross between C57BL/6 and Balb/C (referred to as B6CF1). Eggs were injected with a calculated amount of 2 picograms beta-actin promoter-profilin II-GFP DNA. Founders were identified by polymerase chain reaction using primers specific for the inserted transgene and by Southern blotting with specific probe. Transgenic mice were phenotypically normal.

### REFERENCES

1. M. B. Moser, M. Trommald, T. Egeland, P. Andersen, J Comp Neurol 380, 373-81 (1997); F. Valverde, Exp Brain Res 3, 337-352 (1967); R. G. Coss, A. Globus, Science 200, 787-90 (1978); M. Frotscher, B. Mannsfeld, J. Wenzel, J Hirnforsch 16, 443-50 (1975).
2. K. M. Harris, S. B. Kater, Annu Rev Neurosci 17, 341-371 (1994); F. Crick, Trends Neurosci 5, 44-46 (1982); J. C. Eccles, Naturwiss 66, 147-153 (1979); S. Halpain, Trends Neurosci 23, 141-146 (2000); A. Matus, Science 296, 754-758 (2000).
3. M. Fischer, S. Kaech, D. Knutti, A. Matus, Neuron 20, 847-854 (1998).
4. A. Dunaevsky, A. Tashiro, A. Majewska, C. Mason, R. Yuste, Proc Natl Acad Sci USA 96, 13438-43 (1999).
5. M. Maletic-Savatic, R. Malinow, K. Svoboda, Science 283, 1923-7 (1999); F. Engert, T. Bonhoeffer, Nature 399, 66-70 (1999).
6. B. Lendvai, E. A. Stern, B. Chen, K. Svoboda, Nature 404, 876-881 (2000); M. Fischer, S. Kaech, U. Wagner, H. Brinkhaus, A. Matus, Nat Neurosci 3, 887-894 (2000); Z. Parnass, A. Tashiro, R. Yuste, Hippocampus 10, 561-8 (2000); E. Korkotian, M. Segal, Neuron 30, 751-8. (2001).
7. S. Kaech, M. Fischer, T. Doll, A. Matus, J Neurosci 17, 9565-72. (1997); S. Kaech, H. Parmar, M. Roelandse, C. Bornmann, A. Matus, Proc Natl Acad Sci USA 98, 7086-92. (2001).
8. A. Rao, A. M. Craig, Hippocampus 10, 527-41. (2000).
9. F. M. Smart, S. Halpain, Hippocampus 10, 542-54. (2000).
10. C. H. Kim, J. E. Lisman, J Neurosci 19, 4314-24 (1999).
11. T. Krucker, G. R. Siggins, S. Halpain, Proc Natl Acad Sci US A 97, 6856-6861 (2000).
12. L. Carlsson, L. E. Nystrom, I. Sundkvist, F. Markey, U. Lindberg, J Mol Biol 115, 465-83. (1977).
13. D. Pantaloni, M. F. Carlier, Cell 75, 1007-14. (1993).
14. F. Buss, C. Temm-Grove, S. Henning, B. M. Jockusch, Cell Motil Cytoskeleton 22, 51-61. (1992).
15. M. D. Welch, A. H. DePace, S. Verma, A. Iwamatsu, T. J. Mitchison, J Cell Biol 138, 375-84 (1997); P. J. Goldschmidt-Clermont, L. M. Machesky, S. K. Doberstein, T. D. Pollard, J Cell Biol 113, 1081-9. (1991); I. Lassing, U. Lindberg, Nature 314, 472-4. (1985); I. Lassing, U. Lindberg, J Cell Biochem 37, 255-67. (1988); M. Reinhard et al., Embo J 14, 1583-9. (1995); S. Suetsugu, H. Miki, T. Takenawa, Embo J 17, 6516-26. (1998).
16. F. B. Gertler, K. Niebuhr, M. Reinhard, J. Wehland, P. Soriano, Cell 87, 227-39. (1996).
17. L. M. Machesky, S. J. Atkinson, C. Ampe, J. Vandekerckhove, T. D. Pollard, J Cell Biol 127, 107-15. (1994).
18. N. Wittenmayer, M. Rothkegel, B. M. Jockusch, K. Schluter, Eur J Biochem 267, 5247-56. (2000).
19. B. Honore, P. Madsen, A. H. Andersen, H. Leffers, FEBS Lett 330, 151-5. (1993).
20. W. Witke, J. D. Sutherland, A. Sharpe, M. Arai, D. J. Kwiatkowski, Proc Natl Acad Sci USA 98, 3832-6. (2001).
21. O. Thastrup, P. J. Cullen, B. K. Drobak, M. R. Hanley, A. P. Dawson, Proc Natl Acad Sci USA 87, 2466-70. (1990).
22. L. D. Pozzo-Miller, J. A. Connor, S. B. Andrews, J Physiol 525, 53-61. (2000).
23. K. Maiese, I. Ahmad, M. TenBroeke, J. Gallant, J Neurosci Res 55, 472-85. (1999).
24. G. Y. Wu, K. Deisseroth, R. W. Tsien, Nat Neurosci 4, 151-8. (2001).
25. R. K. Calverley, D. G. Jones, Cell Tissue Res 247, 565-72 (1987); E. G. Gray, Nature 183, 1592-1593 (1959); R. S. Cohen, P. Siekevitz, J Cell Biol 78, 36-48 (1978); Y. Geinisman, F. Morrell, L. de Toledo-Morrell, Brain Res 423, 179-88 (1987); J. Spacek, M. Hartmann, Anat Embryol (Berl) 167, 289-310 (1983).
26. C. Racca, F. A. Stephenson, P. Streit, J. D. Roberts, P. Somogyi, J Neurosci 20, 2512-22. (2000); V. N. Kharazia, R. J. Weinberg, J Comp Neurol 412, 292-302. (1999).
27. W. Witke et al., Embo J 17, 967-76 (1998); A. Lambrechts, J. van Damme, M. Goethals, J. Vandekerckhove, C. Ampe, Eur J Biochem 230, 281-6. (1995).
28. C. Faivre-Sarrailh, J. Y. Lena, L. Had, M. Vignes, U. Lindberg, J Neurocytol 22, 1060-72. (1993).
29. A. Matus, Curr Opin Neurobiol 9, 561-565 (1999).
30. R. S. Petralia et al., Nat Neurosci 2, 31-36 (1999); Y. Takumi, A. Matsubara, E. Rinvik, O. P. Ottersen, Ann N Y Acad Sci 868, 474-82. (1999).
31. A. Rao, E. Kim, M. Sheng, A. M. Craig, J Neurosci 18, 1217-29 (1998).
32. E. Fifkova, R. J. Delay, J Cell Biol 95, 345-50 (1982).
33. A. Matus, M. Ackermann, G. Pehling, H. R. Byers, K. Fujiwara, Proc Natl Acad Sci USA 79, 7590-4 (1982); R. S. Cohen, S. K. Chung, D. W. Pfaff, Cell. Mol. Neurobiol. 5, 271-284 (1985); D. M. Landis, L. A. Weinstein, T. S. Reese, Synapse 1, 552-8 (1987).
34. M. D. Welch, A. Mallavarapu, J. Rosenblatt, T. J. Mitchison, Curr Opin Cell Biol 9, 54-61 (1997).
35. J. A. Markham, E. Fifkova, Brain Res 392, 263-9 (1986).
36. T. V. Bliss, G. L. Collingridge, Nature 361, 31-39 (1993); R. C. Malenka, R. A. Nicoll, Science 285, 1870-4 (1999).
37. C. Luscher, R. A. Nicoll, R. C. Malenka, D. Muller, Nat Neurosci 3, 545-50. (2000).
38. S. J. Martin, P. D. Grimwood, R. G. Morris, Annu Rev Neurosci 23, 649-711. (2000).
39. N. Toni, P. A. Buchs, I. Nikonenko, C. R. Bron, D. Muller, Nature 402, 421-5 (1999).
40. D. E. Shulz, Neuron 28, 25-9. (2000); J. L. McGaugh, Science 153, 1351-8. (1966).
41. U. Frey, R. G. Morris, Trends Neurosci 21, 181-8 (1998).
42. B. Ludin, T. Doll, R. Meili, S. Kaech, A. Matus, Gene 173, 107-11 (1996).

43. G. Banker, K. Goslin, in Culturing Nerve Cells G. Banker, K. Goslin, Eds. (Bradford Books, MIT Press, Cambridge, Mass., 1991) pp. 251-281.

TABLE 1
Enrichment of GFP tagged profilins in dendritic spines.
Percentages of transfected hippocampal neurons showing
spine enrichment of GFP-tagged profilin I and profilin II
or unmodified GFP.

| | Level of enrichment* | | |
|---|---|---|---|
| | ++ | + | − |
| Profilin II (n = 239) | 4% | 52% | 44% |
| Profilin I (n = 68) | — | 12% | 88% |
| GFP (n = 7) | — | — | 100% |

*++, cells showing enrichment in all spines; + cells showing enrichment in some spines; − cells showing no spine enrichment.
(n = total number of cells examined for each construct).

TABLE 2
Dependence of profilin II targeting to dendritic spines on
glutamate receptor subtype.
(a) with glutamate

| | | Number of cells in | | |
|---|---|---|---|---|
| Construct | | category | | |
| Profilin II- | Agonist | ++ | + | − |
| GFP | glutamate | 22 | 3 | 1 |
| GFP | glutamate | — | — | 7 |

(b) with agonists for specific receptor subtypes

| Receptor | | | | |
|---|---|---|---|---|
| Subtype | Agonist | ++ | + | − |
| NMDA | NMDA | 16 | — | — |
| mGluR I | DHPG | 4 | 4 | 1 |
| AMPA | AMPA | — | 2 | 14 |

(c) with glutamate while blocking specific receptor subtypes

| Receptor | | | | |
|---|---|---|---|---|
| Subtype | Antagonist | ++ | + | − |
| NMDA | APV | — | 5 | 17 |
| | MK-801 | — | — | 6 |
| AMPA | CNQX (n = 6)/NBQX (n = 15) | 6 | 14 | 1 |
| | MCPG (n = 17)/Ly341495 | | | |
| mGluR | (n = 9) | 10 | 13 | 3 |
| AMPA and | Ly341495/MCPG/NBQX | 6 | 1 | 1 |
| mGluR | | | | |
| (a) Distribution of profilin II-GFP and GFP in dendrites after stimulating all receptor subtypes by treatment with 10 μM glutamate. Targeting was classified as strong (++), partial (+) and | | | | |
| # no (−) for profilin II-GFP expressing cells. Arrows indicate spines showing strong profilin II targeting, arrowheads point out spines with no accumulation. | | | | |
| (b, c) Profilin II-GFP targeting following 30 min exposure to agonists and antagonists specific for glutamate receptor subtypes. Drug concentrations: NMDA, 10 μM; DHPG, 10 μM; AMPA, 2 μM; APV, 500 | | | | |
| # μM; MK-801, 50 μM, CNQX, 20 μM; NBQX, 20 μM; MCPG, 100 μM-1 mM); Ly341495, 30 μM. n = number of cells examined. | | | | |

TABLE 3
Calcium mediates profilin targeting.
(a). Extracellular Ca²⁺ is required for spine
targeting of profilin II.

| Drug | [Ca²⁺]₀ | ++ | + | − |
|---|---|---|---|---|
| Glutamate | 2 mM | 22 | 3 | 1 |
| Glutamate | 0 | — | — | 5 |
| Thapsigargin | 2 mM | 5 | 5 | 5 |
| Thapsigargin | 0 | | 2 | 5 |

(b). Dependence of profilin II targeting on Ca²⁺ release.

| Drug | ++ | + | − |
|---|---|---|---|
| 100 μM 1S, 3R-ACPD | — | — | 4 |
| 500 μM 1S, 3R-ACPD | 2 | 1 | 2 |
| 1 mM 1S, 3R-ACPD | 8 | — | — |
| Numbers of cells showing accumulation of profilin II-GFP in spines 30 min after different Ca²⁺-releasing treatments. | | | |
| (a). Neurons stimulated with 10 μM glutamate show spine targeting of profilin II only if extracellular Ca²⁺ is present (see also supplementary figure). Thapsigargin (1 μM) also induces extracellular Ca²⁺-dependent spine targeting of profilin II. | | | |
| (b). Release of Ca²⁺ release by the mGluR group I agonist 1S, 3R-ACPD above a threshold | | | |
| # concentration produces a dose-dependent increase profilin II targeting. (++ strong targeting; + weak targeting; − no targeting). | | | |

TABLE 4
Profilin II targeting to spines induced by presynaptic
glutamate release.

| | Numbers of cells | | |
|---|---|---|---|
| Treatment | ++ | + | − |
| 90 mM KCl | 3 | 2 | — |
| 90 mM KCl + 100 μM APV | — | — | 7 |
| Zero [Mg²⁺]₀ | 9 | 2 | 1 |
| Zero [Mg²⁺]_{0 + 100 ng/ml} | — | — | 6 |
| botulinum toxin A | | | |
| Endogenous glutamate release is sufficient to trigger Profilin II targeting to dendritic spines by activating NMDA-receptors. Profilin II-GFP transfected neurons were treated for 30 as follows | | | |
| (a) Depolarization induced by exposing cells to 90 mM KCl 4 times for 3 min with 10 min intervals redistributes profilin II to spines. | | | |
| (b) The same treatment has no effect when NMDA-receptors are blocked by the antagonist APV (100 microM). | | | |
| (c) When external Mg²⁺ is removed from the medium, endogenously released glutamate is sufficient to trigger Profilin II targeting to spines. | | | |
| (d) When cells were pre-incubated with botulinum toxin A (100 ng/ml) for 24 hours to block presynaptic release of glutamate, removal of external Mg²⁺ does not lead to profilin II targeting. As control, subsequent addition | | | |
| # of 5 microM NMDA to directly stimulate postsynaptic receptors leads to targeting. (++ strong targeting; + weak targeting; − no targeting; n = number of cells examined). | | | |

TABLE 5
Enrichment of GFP-tagged gelsolin constructs in dendritic
spines

| | Enrichment (+) | No enrichment (−) |
|---|---|---|
| Gelsolin-GFP (n = 153) | 47% | 53% |
| G2-6-GFP (n = 41) | 52% | 48% |
| GFP (n = 37) | 0% | 100% |

TABLE 6
Receptor subtype specificity of gelsolin
accumulation
a) Treated with agonists for specific receptor subtypes

| Receptor | | | |
|---|---|---|---|
| subtype | Agonist | Accumulation (+) | No change (−) |
| NMDA | NMDA | 11 | 16 |
| mGlu | DHPG | 4 | 7 |
| AMPA | AMPA | 0 | 10 |

(b) Treated with glutamate while blocking specific receptor
subtypes

| Receptor | | | |
|---|---|---|---|
| subtype | Antagonist | Accumulation (+) | No change (−) |
| NMDA | APV | 1 | 14 |
| AMPA | NBQX | 7 | 6 |
| mGlu | MCPG | 3 | 8 |

1-40. (Canceled)

41. A method for identifying or detecting a synapse which has been activated, or assessing the level of activation of a synapse, which method comprises:
(i) determining the presence and\or amount, in a morphologically specialised postsynaptic site in the synapse, of a detectable cellular component associated with the activation, and
(ii) correlating the result of the determination with synaptic activation.

42. A method as claimed in claim 41 wherein the cellular component is a detectable tag or marker the amount of which is increased in activated synapses.

43. A method as claimed in claim 41 wherein the cellular component is present in the neuron cytoplasm and is not detectably present in the synapse prior to activation but localizes into the synapse when the synapse is activated.

44. A method as claimed in claim 41 wherein the determination is a qualitative.

45. A method as claimed in claim 41 wherein the synapse is an excitatory glutamatergic synapse.

46. A method as claimed in claim 41 wherein the determination of the presence and\or amount of the cellular component in the postsynaptic site in the synapse is preceded by exposing the synapse to a putative or known activation stimulus.

47. A method as claimed in claim 46 wherein the activation stimulus is via an NMDA receptor agonist.

48. A method as claimed in claim 41 wherein the postsynaptic site is a dendritic spine.

49. A method as claimed in claim 48 wherein the cellular component is targeted to the spine head.

50. A method as claimed in claim 49 wherein the cellular component is targeted to punctate sites at the surface of the spine head.

51. A method as claimed in claim 41 wherein the cellular component is an endogenous protein or a derivative thereof which includes a detectable label.

52. A method as claimed in claim 51 wherein the endogenous protein is an actin-regulating protein.

53. A method as claimed in claim 52 wherein the actin-regulating protein is selected from: gelsolin, cofilin, Actin Depolymerizing Factor, profilin II.

54. A method as claimed in claim 51 wherein the cellular component is a labelled derivative of an endogenous protein.

55. A method as claimed in claim 54 wherein the labelled derivative is detectable photometrically.

56. A method as claimed in claim 55 wherein the label is selected from: GFP, YFP.

57. A method as claimed in claim 54 wherein the determination of the presence and\or amount of the cellular component in the postsynaptic site in the synapse is preceded by introducing the labelled derivative into one or more of the neurons forming the synapse.

58. A method as claimed in claim 57 wherein the labelled derivative is expressed from nucleic acid encoding therefor introduced into the neuron or a progenitor thereof.

59. A method as claimed in claim 58 wherein the labelled derivative is expressed from an expression construct or vector.

60. A method as claimed in claim 59 wherein the vector is a eukaryotic expression plasmid containing a β-actin promoter

61. A method as claimed in claim 58 wherein the nucleic acid is stably expressed in the neuron.

62. A method as claimed in claim 41 wherein the synapse is present in a cultured spine-bearing hippocampal neuron.

63. A method as claimed in claim 62 wherein a population of synapses is assessed.

64. A method as claimed in claims 58 wherein the synapse is present in, or is extracted from, a non-human transgenic mammal, the cells of which express said labelled derivative.

65. A method as claimed in claim 64 wherein the presence and\or amount of the detectable cellular component in the morphologically specialised postsynaptic site in the synapse is detected in the intact mammal.

66. A method as claimed in claim 64 wherein the presence and\or amount of the detectable cellular component in the morphologically specialised postsynaptic site in the synapse is detected in brain tissue removed from the mammal.

67. A method as claimed in claim 64 wherein the synapse is electrically stimulated in brain tissue removed from the mammal.

68. A method as claimed in claim 64 wherein the synapse is physiologically stimulated in the mammal.

69. A method as claimed in claim 64 wherein the pattern of activated synapses in the brain of the mammal is correlated to a particular disease state.

70. A method for determining whether a synapse or group of synapses are involved in learning and\or memory comprising performing the method of claim 68, wherein the physiological stimulus is involved in learning and/or memory.

71. A method for assessing the ability of an agent to modulate synaptic activation or transmission, comprising the steps of:
(a) contacting the synapse, or neurons forming it, with one or more agents which it is desired to assess,
(b) comparing the activation of the synapse in the presence or absence of said agents by use of the method of claim 61,
(c) optionally, correlating the values obtained in step (b) with the activity of the agent as a modulator.

72. A method as claimed in claim 71 wherein the method includes the steps of:
(i) determining the presence and\or amount, in a morphologically specialised postsynaptic site in the synapse, of the detectable cellular component,
(i bis) exposing the neurons forming the synapse to the agent,
(i ter) measuring the presence and\or amount, in the morphologically specialised postsynaptic site in the synapse, of the detectable cellular component in the presence of the agent,
(ii) comparing the determinations made in (a) and (c).

73. A method as claimed in claim 72 wherein step (i bis) is performed by perfusing the synapse with the agent.

74. A method for assessing the ability of an agent to modulate synaptic activation or transmission, comprising the steps of:
(a) measuring profilin II translocation to actively ruffling membrane in non-neuronal cells,
(b) exposing said cells to the agent,
(c) measuring profilin II translocation to actively ruffling membrane in said cells in the presence of the agent,
(d) correlating an increased value in step (c) compared to step (d) with the ability of the agent to stimulate synaptic activation.

75. A method for screening for compounds for the potential to modulate cognitive function, which method comprises assessing the ability of said compounds to modulate synaptic activation by use of the method of claim 71.

76. A method for screening for compounds for the treatment of epilepsy, neurodegeneration, ischemia, migraine, schizophrenia or depression, which method comprises assessing the ability of said compounds to modulate synaptic activation by use of a method of the method of claim 71.

77. A method for screening for compounds for the potential to modulate cognitive function, which method comprises assessing the ability of said compounds to modulate synaptic activation by use of the method of claim 74.

78. A method for screening for compounds for the treatment of epilepsy, neurodegeneration, ischemia, migraine, schizophrenia or depression, which method comprises assessing the ability of said compounds to modulate synaptic activation by use of a method of the method of claim 74.
